(19) United States
(12) Patent Application Publication
(10) Pub. No.: US 2014/0303545 A1
(43) Pub. Date: Oct. 9, 2014

(54) TAMPON-APPLICATOR ASSEMBLY

(71) Applicant: Caroline Abdon, Helsingborg (SE)

(72) Inventor: Caroline Abdon, Helsingborg (SE)

(21) Appl. No.: 14/355,561

(22) PCT Filed: Nov. 2, 2011
(86) PCT No.: PCT/EP2011/069235

§ 371 Date: Jun. 4, 2014

(51) Int. Cl. A61F 13/26

(52) U.S. Cl. CPC A61F 13/266
USPC 604/14

(57) ABSTRACT

The present invention relates to a tampon-applicator assembly for sanitary application of a tampon. The assembly comprises a tampon having a front end and a rear end, a flexible tubular sheath, and a sleeve. The flexible tubular sheath has a closed rear end, and encloses the tampon such that the rear end of the tampon is directed towards the closed rear end of the sheath, and the sleeve has a front end and a rear end and a longitudinal extension along the tampon and wherein the sleeve is arranged on the outside of the flexible tubular sheath along at least a part of the longitudinal extension of the tampon, whereby at least a portion of the sheath, when a pressing force is applied to the rear end of the sheath towards the front end of the tampon, is arranged to be turned in side out while the tampon is pushed at least partially out of the sleeve. The resent invention further relates to a method of production of the assembly, and the use of the assembly.

### TECHNICAL FIELD

The present invention relates to a tampon-applicator assembly for sanitary application of a tampon, the assembly comprising a tampon, a flexible tubular sheath, and a sleeve.

### BACKGROUND

Tampons are used to give women more freedom during the menstrual period. In use, a tampon needs to be replaced several times a day to avoid odour, bleeding through, or discomfort.

On the market there are two dominant packaging and application methods for tampons: A tampon wrapped in cellophane for application by finger, and a tampon having a cardboard or plastic applicator. Both methods force the user to compromise on hygiene, intimacy and/or comfort. The latter generates undesirable amounts of waste material.

Problems concerned with the applicator-free tampon are primarily related to hygiene: Hands have to be washed with soap and water before and after application, which often is not possible in public conveniences, for example as the result of separate compartments and lavatories often being deficient in soap and water.

Other problems are related to disposal. The electrostatic cellophane packaging components tend to stick to fingers. And they should not be flushed down the toilet and if doing so often remain floating on the flush toilet water surface.

Problems concerned with the applicator tampon are, for example, related to size and disposal. The applicator requires considerable space and cannot be discreetly placed in garment pockets or be discretely concealed in a hand. In an evening out, for example, there is simply not adequate space for the required number of applicator tampons in a small evening purse. As the applicator comprises two plastic or cardboard tubes contained in a package of synthetic resin/cellophane/cardboard, it also produces considerable amounts of waste, thus not being environmentally friendly. Other problems are related to control and comfort. While functioning as an extension of the finger, the applicator is detrimental to control when inserting the tampon. Dry vaginal walls may make it uncomfortable or impossible to insert a new tampon after removing a present one. Accordingly, one has to wait between replacements to allow re-moisture of vaginal walls, particularly at the end of the menstrual period, or after having given birth. The act of inserting a foreign object as an applicator in the vaginal opening may also cause emotional distress to the user.

Further, other problems with applicator tampons are also related to hygiene: While application is more sanitary by using an applicator, one still cannot avoid direct contact with the vaginal opening. The finger tip used when inserting the applicator comes in contact with the tampon. If the tampon is extensively inserted, i.e. until the hand of the user limits further insertion, such that both tubes are fully inserted in the vaginal opening, one has to grasp the proximate tube with two fingers to be capable of removing the applicator. This may possibly be regarded as a negligible detail but institutions concerned with women's health in developing countries, for example, emphasize the importance of fully sanitary applicators.

In a prior art tampon-applicator assembly of the aforesaid kind, disclosed in U.S. Pat. No. 3,749,093A, the sheath is a unitary package-applicator that is turned inside out during application by means of the rear end portion of the sheath being pushed into the remainder of the package for projecting the tampon out of the applicator.

### SUMMARY OF THE INVENTION

An object of the present invention is to overcome problems related to prior art. One object of the present invention is to provide an improved tampon-applicator assembly allowing sanitary application of a tampon.

As used herein, the term contaminant(s) refers to undesired objects, molecules, compositions, bacteria, viruses, or microorganisms which may potentially be harmful to the user of a tampon or other individuals.

According to a first aspect of the present invention, there is provided a tampon-applicator assembly for sanitary application of a tampon. The assembly comprises a tampon having a front end and a rear end, a flexible tubular sheath, and a sleeve, wherein the flexible tubular sheath has a closed rear end, and encloses the tampon such that the rear end of the tampon is directed towards the closed rear end of the sheath, and the sleeve has a front end and a rear end and a longitudinal extension along the tampon and wherein the sleeve is arranged on the outside of the flexible tubular sheath along at least a part of the longitudinal extension of the tampon, whereby at least a portion of the sheath, when a pressing force is applied to the rear end of the sheath towards the front end of the tampon, is arranged to be turned inside out while the tampon is pushed at least partially out of the sleeve.

Thus, the flexible tubular sheath provides enclosure and thus protection of the tampon from contaminants. It is also adapted to be turned inside out from an opened front end thereof. Thus, the assembly according to the invention provides both control and sanity; by the sleeve that imparts the structural capability of controllably guiding of the tampon, and, by the sheath that provides a sanitary shield both as a package as well as during application.

The closed rear end of the sheath provides sanity as it prevents direct contact with the tampon during application. Further, the closed rear end of the sheath provides sanity as it prevents contamination of the tampon, for example during storage in a pocket or purse.

The sleeve efficiently provides protection and stability to the tampon during storing, for example, as it may prevent the tampon from swelling due to moisture in surrounding air.

The longitudinal extension of the sleeve results in that the assembly when ready for application of the tampon, or positioning of the tampon inside the vagina, may efficiently be held on to a single finger of the user. Thus, it further results in efficient one hand handling of the tampon during application of the tampon.

The sleeve being arranged on the outside of the flexible tubular sheath results in sanitary application of the tampon.

The feature that at least a portion of the sheath is arranged to be turned inside out while the tampon is pushed at least partially out of the sleeve results in a sanitary handling and application of the tampon as the surface of the sheath originally being on the outside and possibly contaminated is turned into the inside facing the finger of the user or the outer surface of the sleeve, thus not risking of getting in contact with the vagina during application of the tampon.

According to one embodiment of the invention, the sheath may have a longitudinal extension exceeding the length of the tampon. Thereby the sheath may cover the full length of the sleeve and a part of the finger of the user during application of the tampon. Further, the sheath may totally enclose the tampon to minimise the risk of contaminating the tampon.

According to one additional embodiment, for the case with the sheath having a longitudinal extension exceeding the length of the tampon, the sheath may have a seal in its front end in a position in front of the front end of the tampon. Thereby, the sheath may fully and hermetically enclose the tampon, thus creating protection from contamination and moist prior to use. The seal being positioned in front of the tampon provides for efficient opening of the sheath by the seal.

The extension of the sheath exceeding the length of the tampon further provides for the possibility of; after having made access to the tampon, for the case with the assembly comprising a seal, by opening the seal for example by tearing or pushing the tampon out through the seal; pulling the sheath down in a direction towards the rear end of the tampon over the frontal edge of the sleeve, thus turning, at least a portion of, the sheath inside out.

It is understood that the assembly must not comprise a seal. If the assembly does not comprise a seal, the assembly may be enclosed by a second protective packing or wrapping, such as a cellophane packing or wrapping. Such a second protective packing further provides protection from moisture reaching the tampon and thus prevents swelling of the tampon during storing.

According to one embodiment of the invention, the assembly is fully enclosed in a wrapping or a second sheath.

According to one embodiment, the sleeve may be radially expandable. This can e.g. be made by providing the sleeve with a longitudinal slit. Thus, one sleeve, or one size of the sleeve, may efficiently be used for several different sizes of tampons, still providing a grip around the tampon. Further, a single sleeve may fit different sizes of fingers or pistons.

According to one embodiment, the sleeve may be longitudinally rigid.

According to one embodiment, the sleeve may be radially expandable and longitudinally rigid.

According to one embodiment, the tampon may project from the front end of the sleeve prior to and during use of the assembly.

According to one embodiment of the invention, the full extension of the sheath may be arranged to be turned inside out during application of the tampon. Thus, efficient sanitary storing and application of the tampon may be ensured.

According to one embodiment of the invention, the longitudinal extension of the sheath may be adapted to allow the front end of the sheath to extend over the front end of the sleeve towards a rear end of the sleeve along an exterior wall portion of the sleeve. Allowing the front end of the sheath to extend over the front end of the sleeve towards a rear end of the sleeve along an exterior wall portion of the sleeve is an efficient way of turning the sheath inside out when the assembly is prepared for use. Further, the assembly may efficiently be made ready for use, by the user pushing a finger, or for example a piston, against the rear end of the tampon and into the sleeve, thus pushing the tampon slightly further into the sleeve, followed by or simultaneously with pulling/moving the front end of the sheath in a direction from the front end of the tampon towards the rear end of the sleeve such that the tampon will further project out of the front end of the sleeve during turning inside out of the sleeve.

According to one embodiment, the sheath may have has a seal in at least one position selected from the group of: behind the rear end of the tampon; in a position between the front end of the tampon and the rear end of the tampon; and in a position in front of the front end of the tampon. A seal in front of the front end of the tampon may provide for efficient and sanitary sealing of the assembly. The seal being in a position behind the rear end of the tampon may be yet another way for efficient and sanitary sealing of the assembly. In the case of the seal being in a position behind the rear end of the tampon or between the front end of the tampon and the rear end of the tampon a seal may in addition be provided in front of the front end of the tampon.

According to one embodiment, the front end of the sheath may be arranged to extend over the front end of the sleeve towards a rear end thereof along an exterior wall portion of the sleeve forming a fold along the exterior wall portion, whereby the front end of the sheath is directed towards the front end of the sleeve. By such an arrangement of the sheath, all of the exterior surface of the sheath exposed to the ambience prior to use of the assembly may be turned inside out when the assembly is used, thus minimising risks of contamination and ensuring a sanitary application of the tampon. It is understood that said fold must not be a sharp fold but that the use of the term “fold” herein is intended for giving a geometrical description, thus including soft turns of the sheath or other geometries creating a change of direction taken by the sheath.

According to one embodiment, the seal may be releasable. Thus, without the use of instruments such as scissors, the seal may be released to provide access to the tampon and to make the assembly ready for use.

Depending on, for example, the longitudinal extension of the sheath, the seal may be arranged adjacent or in contact with the rear end of the sleeve or the tampon; or the front end of the sleeve or the tampon. The seal may for example be prepared by gluing, taping or welding the sheath. The seal may also be prepared, particularly with regards to the seal in a position between the front end of the tampon and the rear end of the tampon, by a band, thread, rubber band or piece of tape, or similar, around the circumference of the tampon. The seal may be arranged for breaking or bursting open by the front end of the tampon when it is pushed out of the sleeve. The seal may be tearable, for example by means of a perforated or weakened tear line.

According to one embodiment, the tampon may be fully enclosed by the sheath(s), preferably hermetically enclosed by the sheath. Thus, risks of contaminating the tampon prior to use is minimised.

According to one embodiment, the sheath may have at least two seals.

According to an additional embodiment wherein the sheath may have at least two seals, a first seal may be in a position in front of the front end of the tampon, and a second seal may be in a position behind the rear end of the tampon or in a position between the front end of the tampon and the rear end of the tampon. Thus, risks of contaminating the tampon prior to use is minimised. Further, the first seal may ensure that no contaminants reaches the tampon, while the second seal ensures that no contaminants, such as dirt or dust, collects under the sheath.

According to one embodiment, a removal string of the tampon may be releasably attached to the closed rear end of the sheath or to an interior wall portion of the sheath, preferably adjacent the closed rear end thereof. Thus, the removal string may efficiently be correctly positioned after application of the tampon, and risks of the string to be curled up inside the vagina is minimised.

The removal string may in addition be non-fixed to the sheath. It may be positioned between the tampon and the sheath in the assembly, attached only to the rear end of the tampon. Thus, when the assembly is positioned on, for example, the finger of the user the removal string may efficiently be positioned along the finger on the outside of the sheath.

According to one embodiment, the sheath may be adapted to be slidable in relation to the front end of the sleeve and in relation to an inner surface of the sleeve. Thus, the sheath may easily be pulled down in a direction towards the rear end of the sleeve and thus efficiently be turned inside out during positioning the assembly on a finger of the user or a piston.

According to one embodiment, the sleeve may have a length corresponding to at least 50% of the length of the tampon, preferably at least 75% of the length of the tampon, for example at least 100% of the length of the tampon or essentially 100% of the length of the tampon. Such lengths of the sleeve ensures that the assembly is securely positioned on the finger of the user during application of the tampon, as well as ensuring that the tampon is secured to the sleeve during the application. Further such a length ensures that the tampon may be applied using only one hand of the user.

According to one embodiment, the pressing force may be applied by a piston adapted to cooperate with said sleeve, or a finger of a user of the applicator. The sleeve is arranged such that the finger or the piston shall be inside the sleeve. Thus, the sleeve may have an inner diameter which is larger than or similar to the outer diameter of the piston or the finger when the assembly is in use. In the case where the sleeve is radially expandable, i.e. flexible, the inner diameter of the sleeve may, before the finger or piston has been inserted, be smaller than the diameter of the finger or the piston.

According to one embodiment, the force may be directed in a direction from the rear end of the tampon towards the front end of the tampon. Thus, efficient insertion of the finger or the piston into the sleeve is enabled, and the rear end of the tampon is pushed into or further into the sleeve in a direction towards the front end of the sleeve.

According to one embodiment, the pressing force may further be combined with a pulling force applied on said sheath, said pulling force being adapted to be applied by the user of the applicator. Thus, the tampon may efficiently be prepared for application of the tampon by positioning and pressing the tip of the finger or the piston against the closed rear end of the sheath and pulling the sheath in a direction from the front end towards the rear end of the sleeve and, optionally depending on the length of the sheath, down over the finger, during which procedure the finger or piston is forwarded into the sleeve and the tampon is pushed partly out of the sleeve by said finger or piston, while the sheath is turned inside out and pulled down over the finger or piston, thus protecting the finger or piston from coming in contact with the vagina during positioning of the tampon. Thus, the pulling force may be directed in a direction from the front end of the tampon towards the rear end of the tampon

According to a second aspect of the invention, there is provided a method for production of the assembly according the invention and embodiments of the invention, comprising the steps of: providing a tampon; providing a sheath material; positioning the tampon in contact with the sheath material; enclosing the tampon by the sheath material; providing a sleeve;

positioning the enclosed tampon in the sleeve; optionally folding the sheath; and optionally sealing the sheath.

According to a third aspect of the invention, there is provided a use of the assembly according to the first aspect of the invention.

With regards to the second and third aspects of the invention, the discussions and embodiments of the first aspect of the invention are directly applicable whereby references to these discussions and embodiments are made.

### BRIEF DESCRIPTION OF THE DRAWINGS

FIG. 1a is a schematic side view of a tampon-applicator assembly according to an embodiment of the invention.

FIG. 1b is a schematic top view of the tampon-applicator assembly illustrated in FIG. 1a.

FIG. 2a is a schematic cross-sectional view of the tampon-applicator assembly according to an embodiment of the invention.

FIG. 2b is a schematic cross-sectional view of the tampon-applicator assembly illustrated in FIG. 2a during positioning on a finger of a user.

FIG. 2c is a schematic cross-sectional view of the tampon-applicator assembly illustrated in FIGS. 2a and 2b positioned on a finger of a user and ready for use.

FIGS. 3a to 3j are schematic cross-sectional views of tampon-application assemblies according to different embodiments of the invention.

FIGS. 4a to 4d are side views of the tampon-applicator assemblies according to embodiments of the invention.

### DETAILED DESCRIPTION OF EMBODIMENTS

The following detailed descriptions of embodiments of the invention are included as examples of embodiments of the invention to improve the understanding of the invention and should not in any way be interpreted as limiting the scope of the invention. It is realised that even if a finger is illustrated and discussed with regards to the figures, also, for example, a piston may be used instead of the finger.

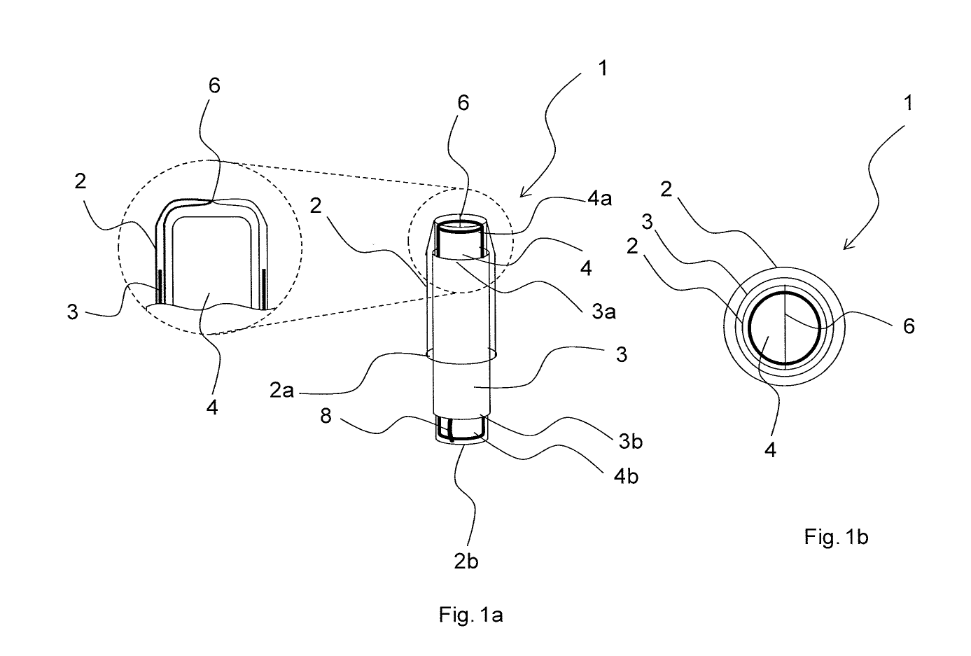

With reference to FIGS. 1a and 1b one embodiment will now be discussed. FIG. 1a is a perspective view of a tampon-applicator assembly 1, and FIG. 1b is a top view of the assembly 1. The inset is a cross-sectional view of the front part of the assembly 1. The assembly 1 has a tampon 4, a sleeve 3 and a sheath 2, which in this example is transparent, thus material enclosed by the sheath 2 can be viewed through the sheath 2. The tampon is for vaginal use eg. during menstruation. The sheath 2 has a seal 6 in front of the front end of the tampon 4a, and a closed rear end 2b, adjacent to the rear end of the tampon 4b. In this example the seal 6 is made by heat sealing across the longitudinal extension of the sheath 2. The sheath 2 has a front end 2a extending over the front end of the sleeve 3a towards the rear end of the sleeve 3b. The tampon 4 has a removal string 8 attached to its rear end 4b and positioned along the side of the tampon 4, the removal string 8 only being partly visible in FIG. 1a, while most of the removal string 8 is obscured by the sleeve 3. In this example the tampon 4 has a length of approximately 5 cm and a diameter of approximately 1 cm. The sleeve 3 has a length of approximately 4 cm or 80% the length of the tampon.

The flexible tubular sheath according to the invention may be made of any suitable material, such as a material selected from the group comprising plastics, polymers, latex, elastomers, and rubber.

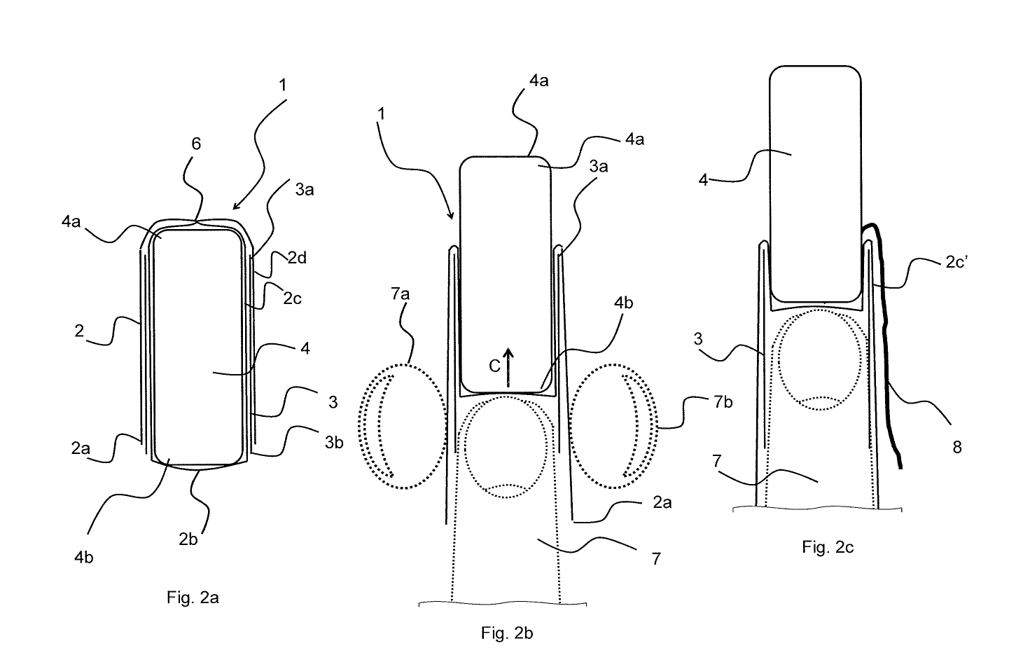

With reference to FIGS. 2a, 2b, and 2c, embodiments of the present invention will now be discussed: FIGS. 2a, 2b, and 2c are schematic cross- sectional illustrations of the assembly 1 through the centre of the assembly 1. FIG. 2a illustrates the assembly 1 prior to use, such as when carried in a purse of the user: The assembly 1 has a flexible tubular sheath 2, with a closed rear end 2b. The sheath 2 is sealed by means of a seal 6, positioned in front of the front end of the tampon 4a, preventing contaminants from reaching the tampon 4. In this example the seal 6 is in the form of a heat seal made by pressing and heating across the flexible tubular sheath 2. The tampon 4, having a front end 4a and a rear end 4b, is fully enclosed by the sheath 2, due to the seal 6 and the closed rear end 2b of the sheath. On the outside of the sheath 2 is arranged the sleeve 3, which in this example is a plastic tube with an opening along its full length (opening not illustrated), thus being radially expandable. The thus radially expandable sleeve 3 may as one result be used for several different radii of tampons and different sizes of fingers of the user. In this example, the front end of the sheath 2a proceeds downwards from the seal 6 over the front end of the sleeve 3a towards a rear end of the sleeve 3b along an exterior wall portion of the sleeve in a direction towards the rear end of the tampon 4b. It is realised that the front end of the sheath 2a alternatively could have been positioned on one side of the assembly 1, as a flap, in which case the seal 6 might have been omitted. The sheath 2 has an inner part 2c positioned on the inside of the sleeve 3 and an outer part 2d positioned on the outside of the sleeve 3. As will be evident below, the inner part 2c of the sheath 2 will be turned inside out during use of the assembly 1. Thus, in this particular example approximately 50% of the sheath 2 will be turned inside out during use, thus producing an uncontaminated surface of the part of the assembly closest to the tampon, when the assembly is ready for use.

With reference to FIGS. 2b and 2c the use of the assembly will now be discussed. One suitable example of preparing the assembly 1 for application of the tampon, or positioning of the tampon in the vagina, is by the user positioning the tip of her finger 7 of one hand against the closed rear end 2b of the sheath 2, while the user uses fingers 7a and 7b of her other hand to grip the assembly 1. By pulling the sheath with fingers 7a and 7b in a direction towards the rear end of the sleeve 3b and executing a pressing force by finger 7 on the rear end of the tampon 4b in a direction indicated by arrow C; the tampon 4 will be sliding and projecting out of the front end of the sleeve 3a while simultaneously the inner part 2c of the sheath 2 will be turned inside out when sliding over the front edge of the sleeve 3, and the outer part 2d of the sheath 2 will be pulled down over the finger 7 of the user. In this example the tampon 4 itself breaks the seal 6 when projecting out from the sleeve. The seal may also be opened by the user, for example by tear opening the seal. The closed rear end of the sheath 2 prevents the finger 7 from contaminating the tampon 4 as well as preventing the tampon 4 for being contaminated for example when carried in a purse.

FIG. 2c illustrates the assembly when ready for use by insertion of the tampon into the vagina. The major part of the inner part 2c of the sheath 2 have been turned inside out (now indexed by 2c′ in FIG. 2c), thus providing an uncontaminated surface on the outside of the sleeve minimising the risk of introducing contaminants into the vagina. At least a major part of the outer part 2d of the sheath has been pulled over the finger 7 of the user, thus providing a sanitary environment for the finger 7 and avoiding vaginal contact. A removal string 8 (not illustrated in FIGS. 2a and 2c), attached to the rear end of the tampon 4b, is hanging alongside the finger 7. Prior to having made the assembly 1 ready for use, i.e. as illustrated in FIG. 2a, the removal string 8 was positioned alongside the tampon 4. It should be noted that a smaller part of the tampon 4 still is inside the sleeve 3, while the finger 7 of the user occupies a major part of the interior of the sleeve 3, thus the assembly is secured to the finger of the user in a stable fashion while still holding on to the tampon 4. At this stage the assembly may be handled by a single hand of the user, i.e. the hand holding the finger 7. When inserted inside the vagina, the tampon can easily be released from the assembly, for example by bending the finger slightly; or by exerting more force on the rear end of the tampon 4b, for example by pulling the free end of the sheath 2.

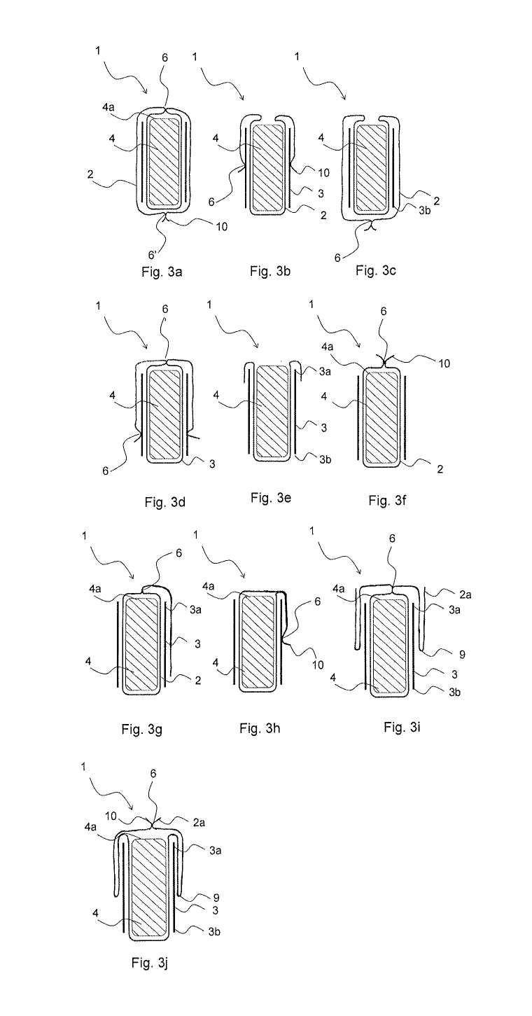

With reference to FIGS. 3a to 3h, different embodiments will now be discussed, the different embodiments differing with regards to the sheath 2 and the seal(s) 6. The FIGS. 3a to 3h are schematic cross-sectional views through the centre of the assembly. It is realised that even if a distance between the tampon 4, the sheath 2, and/or the sleeve 3 is illustrated it is for the clarity of the illustrations and in reality the distance may not exist or be negligible

FIG. 3a illustrates the assembly 1 with a tampon 4, wherein the sheath 2 is sealed by means of a seal 6 in front of the front end of the tampon 4a, wherein the sheath 2 further extends from the seal 6 along the full length of the tampon to a second seal 6′ positioned below the rear end of the sleeve 3b and the rear end of the tampon 4b. Such an assembly with two seals provides extra protection from contamination of the tampon 4 prior to use. The seal 6′ is, optionally, provided with one or two flap(s) 10, which flap(s) 10 makes opening the seal 6′ more efficient.

FIG. 3b illustrates the assembly 1 with a tampon 4, wherein the sheath 2 is sealed by means of a seal 6 around the sleeve 3. Such a seal 6 may be manufactured for example by heat sealing or by an adhering the sheath against the sleeve 3 by means of an adhesive. The open end of the sheath 2 proceeding from the seal 6 provides a flap 10 which may be grabbed by the user thus facilitating opening of the assembly 1. The sheath 2 of the illustrated assembly 1 may protect the finger of the user during positioning of the tampon 4. The arrangement of the sheath 2 with an opening in front of the tampon 4 does not provide for a total or hermetical enclosing of the tampon 4, and in order to provide a sanitary environment for the tampon 4 the assembly 1 could, for example, be enclosed by a wrapping (not illustrated).

FIG. 3c illustrates the assembly 1 with a tampon 4, wherein the sheath 2 is sealed by means of a seal 6 below the rear end of the sleeve 3b. When the assembly is positioned on the finger 7 of a user and ready for application, the sheath will proceed further down the finger 7 compared to the sheath 2 of according to the embodiment illustrated in FIG. 3b.

FIG. 3d illustrates the assembly 1 with a tampon 4, similar to the assembly illustrated in FIG. 3b to which references are made, with the exception of a second seal 6′ in front of the front end of the tampon 4a, thus providing a total enclosing of the tampon, and with the exception of the sheath 4 being longer and proceeding further down the sleeve 3.

FIG. 3e illustrates the assembly 1 with a tampon 4, wherein the sheath 2 has a longitudinal extension exceeding the length of the tampon and the front end of the sheath 2a extends over the front end of the sleeve 3a towards a rear end of the sleeve 3b along an exterior wall portion of the sleeve 3. In this example, the assembly is seal-less. Such an assembly, although not benefitting from a hermetical or total enclosure of the tampon, provides a protection of the finger 7 of the user when ready for use.

FIG. 3f illustrates the assembly 1 with a tampon 4, wherein the sheath 2 has a longitudinal extension exceeding the length of the tampon and a seal 6 in a position in front of the front end of the tampon 4a, with optional flap(s) 10. Such an assembly benefits from a hermetical or total enclosure of the tampon and provides a protection of the finger 7 of the user when ready for use.

FIG. 3g illustrates the assembly 1 with a tampon 4, and a seal 6 in a position in front of the front end of the tampon 4a, thus providing a total enclosure of the tampon 4. The front end of the sheath does not proceed from the seal 6 in an inside out fashion over the sleeve 3 but has been positioned down one side of the sleeve 3. Optionally, the open end of the sheath 2 could be adhered to the sleeve 3. When the user prepares such an assembly to be used, the user may suitably turn the open end of the sheath 2 inside and out over the front end of the sleeve 3a.

FIG. 3h illustrates the assembly 1 with a tampon 4, similar to the assembly discussed with references to FIG. 3g, with the exception of the seal 6 being in a position alongside the sleeve 3 instead of in front of the front end of the tampon 4a, and the positioning of the optional flap(s) 10.

FIG. 3i illustrates an assembly 1 with a tampon 4, wherein he sheath is sealed by means of a seal 6 in front of the front end of the tampon 4a, and wherein the front end of the sheath 2a extends over the front end of the sleeve 3a towards the rear end of the sleeve 3b along an exterior wall portion of the sleeve 3 forming a fold 9 along the exterior wall portion, whereby the front end of the sheath 3a is directed in a direction from the rear end of the sleeve 3b to the front end of the sleeve 3a. Such an assembly 1 benefits from having a major part of the sheath 2 turned inside out when used.

FIG. 3j illustrates an assembly 1 with a tampon 4, wherein the front end of the sheath 2a extends over the front end of the sleeve 3a towards the rear end of the sleeve 3b along an exterior wall portion of the sleeve 3 forming a fold 9 along the exterior wall portion, whereby the part of the sheath after the fold is directed towards the front end of the tampon 4a where it forms a releasably closed front end by means of a seal positioned in front of the front end of the tampon 4a, optionally with at least one flap 10 for efficient tearing or opening of the seal. Such an assembly benefits from that essentially the entire length of the sheath 2 is turned inside out when used, thus providing an efficient protection against contamination of the tampon when handled or stored and of the vagina during positioning of the tampon, as well as providing efficient protection of the finger 7 of the user during positioning of the tampon 4.

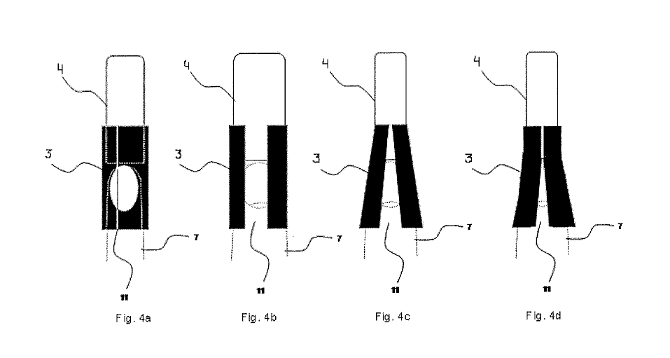

With references to FIGS. 4a to 4d, the assembly with radially expandable sleeves will now be discussed. FIGS. 4a to 4d illustrates schematically the assembly ready for use. To improve the clarity, only the tampon 4, the sleeve 2, and the finger 7 of the user are illustrated together with an opening 11 in the sleeve in the form of a slit, which opening makes the sleeve 2 radially expandable. It is realised that the sleeve 2 may be made radially expandable in other ways, such as being made out of a flexible material such as rubber, or such as having a cross-section in the shape of a star. The exemplified sleeve 3 is radially expandable, but essentially not expandable in the longitudinal direction, or rigid in the longitudinal direction. FIG. 4a illustrates the sleeve in a semi-transparent fashion such that the outline of the finger 7 of the user is visible through the sleeve 2. In this example the sleeve is not radially expanded and in this form it suitably holds a normal sized tampon 4 onto a normal sized finger 7 of the user. The same sleeve is illustrated if FIG. 4b, wherein it is radially expanded, as indicated by a wider opening 11 of the slit, to suitably hold a larger sized tampon onto a larger finger 7 of a user. In FIG. 4c it is illustrated that the same sleeve 2 suitably may hold a smaller sized tampon onto a large sized finger 7 of the user. In this case the sleeve is expanded more at its rear end as compared to its front end. For such a purpose and according to another embodiment of the invention the sleeve 2 may have a frustoconical shape provided with a longitudinal slit.

Yet another embodiment is disclosed in FIG. 4d. In this embodiment a portion of the sleeve 3 closest to the rear end of the tampon 4b is cylindrical while the other portion of the sleeve has a frustoconical shape. The sleeve 3 is radially expandable by means of a longitudinal slit. The cylindrical portion of the sleeve 3 efficiently holds the tampon 4 when the assembly is ready for use while the frustoconical portion allows insertion of the finger of a user. For example, 25% of the length of the sleeve 3 may be cylindrical and 75% frustoconical.

To simplify the understanding FIGS. 4a-4d do not illustrate the sheath.

It will be appreciated that the present invention is not limited to the disclosed embodiments. A number of modifications and variants are possible and the invention is consequently defined exclusively by the appended claims.

1. A tampon-applicator assembly for sanitary application of a tampon, the assembly comprising a tampon having a front end and a rear end, a flexible tubular sheath, and a sleeve, wherein
the flexible tubular sheath has a closed rear end, and encloses the tampon such that the rear end of the tampon is directed towards the closed rear end of the sheath, and
the sleeve has a front end and a rear end and a longitudinal extension along the tampon and wherein the sleeve is arranged on the outside of the flexible tubular sheath along at least a part of the longitudinal extension of the tampon, whereby
at least a portion of the sheath, when a pressing force is applied to the rear end of the sheath towards the front end of the tampon, is arranged to be turned inside out while the tampon is pushed at least partially out of the sleeve.

2. The assembly according to claim 1, wherein the sheath has a longitudinal extension exceeding the length of the tampon.

3. The assembly according to claim 2, wherein the sheath has a seal in its front end in a position in front of the front end of the tampon.

4. The assembly according to claim 1, wherein the sleeve is radially expandable.

5. The assembly according to claim 1, wherein the longitudinal extension of the sheath is adapted to allow the front end of the sheath to extend over the front end of the sleeve towards a rear end of the sleeve along an exterior wall portion of the sleeve.

6. The assembly according to claim 5, wherein the sheath has a seal in at least one position selected from the group of: behind the rear end of the tampon; in a position between the front end of the tampon and the rear end of the tampon; and in a position in front of the front end of the tampon.

7. The assembly according to claim 1, wherein the front end of the sheath is arranged to extend over the front end of the sleeve towards a rear end thereof along an exterior wall portion of the sleeve forming a fold along the exterior wall portion, whereby the front end of the sheath is directed towards the front end of the sleeve or is positioned in front of the front edge of the sleeve.

8. The assembly according to claim 6, wherein the seal is releasable.

9. The assembly according to claim 1, wherein the tampon is fully enclosed by the sheath, preferably hermetically enclosed by the sheath.

10. The assembly according to claim 1, wherein the sheath has at least two seals,
a first seal in a position in front of the front end of the tampon, and
a second seal in a position behind the rear end of the tampon or in a position between the front end of the tampon and the rear end of the tampon.

11. The assembly according to claim 1, wherein a removal string of the tampon is releasably attached to the closed rear end of the sheath or to an interior wall portion of the sheath, preferably adjacent the closed rear end thereof

12. The assembly according to claim 1, wherein the sheath is adapted to be slidable in relation to the front end of the sleeve and in relation to an inner surface of the sleeve.

13. The assembly according to claim 1, wherein the sleeve has a length corresponding to at least 50% of the length of the tampon, preferably at least 75% of the length of the tampon.

14. The assembly according to claim 1, wherein the pressing force is applied by a piston adapted to cooperate with said sleeve, or by a finger of a user of the applicator.

15. The assembly according to claim 1, wherein the pressing force further is combined with a pulling force applied on said sheath, said pulling force being adapted to be applied by the user of the applicator.

16. A method for production of the assembly according to claim 1, comprising the steps of:
providing a tampon;
providing a sheath material;
positioning the tampon in contact with the sheath material;
enclosing the tampon by the sheath material;
providing a sleeve;
positioning the enclosed tampon in the sleeve;
optionally folding the sheath; and
optionally sealing the sheath.

17. A use of the assembly according to claim 1.
